SEQUENCE LISTING (9 sequences)

SEQ ID NO: 1 - protein, 188 aa, Nocardiopsis sp.
  mat_peptide (1)..(188)
ADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 2 - protein, 188 aa, Nocardiopsis dassonvillei
  mat_peptide (1)..(188)
ADIIGGLAYYMGGRCSVGFAATNSAGQPGFVTAGHCGTVGTGVTIGNGTGTFQNSVFPGNDAAFVRGTSNFTLTNLVSRYNSGGYQSVTGTSQAPAGSAVCRSGSTTGWHCGTIQARNQTVRYPQGTVYSLTRTNVCAEPGDSGGSFISGSQAQGVTSGGSGNCSVGGTTYYQEVTPMINSWGVRIRT

SEQ ID NO: 3 - protein, 188 aa, Nocardiopsis alba
  mat_peptide (1)..(188)
ADIIGGLAYTMGGRCSVGFAATNASGQPGFVTAGHCGTVGTPVSIGNGQGVFERSVFPGNDSAFVRGTSNFTLTNLVSRYNTGGYATVSGSSQAAIGSQICRSGSTTGWHCGTVQARGQTVSYPQGTVQNLTRTNVCAEPGDSGGSFISGSQAQGVTSGGSGNCSFGGTTYYQEVNPMLSSWGLTLRT

SEQ ID NO: 4 - protein, 188 aa, Nocardiopsis prasina
  mat_peptide (1)..(188)
ADIIGGLAYTMGGRCSVGFAATNAAGQPGFVTAGHCGRVGTQVSIGNGQGVFEQSIFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGNQAQGVTSGGSGNCRTGGTTFYQEVTPMVNSWGVRLRT

SEQ ID NO: 5 - protein, 188 aa, Nocardiopsis prasina
  mat_peptide (1)..(188)
ADIIGGLAYTMGGRCSVGFAATNAAGQPGFVTAGHCGRVGTQVTIGNGRGVFEQSIFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGNQAQGVTSGGSGNCRTGGTTFYQEVTPMVNSWGVRLRT

SEQ ID NO: 6 - protein, 196 aa, Artificial Sequence
  Synthetic construct
ADIIGGLAYYMGGRCSVGFAATNASGQPGFVTAGHCGTVGTPVSIGNGKGVFERSIFPGNDSAFVRGTSNFTLTNLVSRYNSGGYATVAGHNQAPIGSAVCRSGSTTGWHCGTIQARNQTVRYPQGTVYSLTRTTVCAEPGDSGGSYISGTQAQGVTSGGSGNCSAGGTTYYQEVNPMLSSWGLTLRTQSHVQSAP

SEQ ID NO: 7 - protein, 8 aa, Artificial Sequence
  Synthetic construct
QSHVQSAP

SEQ ID NO: 8 - protein, 269 aa, Thermomyces lanuginosus [sequence omitted]

SEQ ID NO: 9 - protein, 478 aa, Aspergillus oryzae
  mat_peptide (1)..(478)
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

(19) United States
(12) Patent Application Publication
(10) Pub. No.: US 2011/0158976 A1
(43) Pub. Date: Jun. 30, 2011

(54) Enzymes for pharmaceutical use

(75) Inventors: Allan Svendsen, Horsholm (DK); Svend Kaasgaard, Skovlunde (DK); Kim Borch, Birkerod (DK); Morten Fischer, Vedbaek (DK); Dan Pettersson, Lynge (DK); Peter Colin Gregory, Hannover (DE)

(73) Assignee: NOVOZYMES A/S, BAGSVAERD (DK)

(21) Appl. No.: 11/597,273

(22) PCT Filed: May 24, 2005
(86) PCT No.: PCT/DK05/00342

§ 371 Date: Nov. 20, 2006

Related U.S. Application Data
(60) Provisional application No. 60/574,742, filed on May 27, 2004.
(60) Provisional application No. 60/645,477, filed on Jan. 20, 2005.

(30) Foreign Application Priority Data
May 24, 2004 (DK) PA2004 00810
Jan. 20, 2005 (DK) PA 2005 00101

(51) Int. Cl. A61K 38/48; C12N 9/52; A61P 3/00

(52) U.S. Cl. 424/94.63; 435/220

(57) ABSTRACT

The pharmaceutical use of proteases related to a protease derived from Nocardiopsis sp. NRRL 18262 (SEQ ID NO: 1), optionally in combination with a lipase and/or an amylase. Examples of medical indications are: Treatment of digestive disorders, pancreatic exocrine insufficiency (PEI), pancreatitis, cystic fibrosis, diabetes type I, and/or diabetes type II.

### TECHNICAL FIELD

The present invention relates to the pharmaceutical use of proteases related to a protease derived from Nocardiopsis sp. NRRL 18262 (SEQ ID NO: 1), optionally in combination with a lipase and/or an amylase. Examples of medical indications are: Treatment of digestive disorders, pancreatic exocrine insufficiency (PEI), pancreatitis, cystic fibrosis, diabetes type I, and/or diabetes type II.

### BACKGROUND ART

Several commercial medicaments in the form of pancreatic enzyme supplements are known for the treatment of pancreatic exocrine insufficiency. The active ingredients of these products are digestive enzymes, mainly amylase, lipase and protease, which are normally produced in the pancreas and excreted to the upper part of the small intestine (the duodenum). The enzymes used in such medicaments derive from bovine or swine pancreas.

U.S. Pat. No. 5,614,189 (EP 600868) describes the use of certain microbial lipases in pancreatic enzyme replacement therapy, for example in the treatment of patients suffering from cystic fibrosis.

WO 00/54799 describes the use of enzyme mixtures having lipolytic, proteolytic and amylolytic activity in the treatment of diabetes mellitus type I and I.

WO 02/060474 describes the use of certain lipases, proteases and amylases in the treatment of mal-digestion.

The protease derived from Nocardiopsis sp. NRRL 18262 (SEQ ID NO: 1), as well as its preparation and various industrial applications thereof are described in WO 88/03947 and WO 01/58276.

### SUMMARY OF THE INVENTION

The present invention relates to a protease of at least 70% identity to SEQ ID NO: 1, for use as a medicament, optionally in combination with a lipase, and/or an amylase.

The invention also relates to the use of such proteases for the manufacture of a medicament for the treatment of digestive disorders, PEI, pancreatic insufficiency, pancreatitis, cystic fibrosis, diabetes type I, and/or diabetes type II, these uses optionally further comprising the use of a lipase, and/or an amylase.

The invention furthermore relates to a pharmaceutical composition comprising such proteases, together with at least one pharmaceutically acceptable auxiliary material, optionally including a lipase and/or an amylase.

The invention also relates to a method for the treatment of digestive disorders, PEI, pancreatic insufficiency, pancreatitis, cystic fibrosis, diabetes type I, and/or diabetes type II, by administering a therapeutically effective amount of such proteases, optionally together with a lipase and/or an amylase.

### DETAILED DESCRIPTION OF THE INVENTION

### Enzymes

The term “protease” is defined herein as an enzyme that hydrolyses peptide bonds. It includes any enzyme belonging to the EC 3.4 enzyme group (including each of the thirteen subclasses thereof, these enzymes being in the following referred to as “belonging to the EC 3.4 . . . group”). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

The present invention relates to the pharmaceutical use of proteases of at least 70% identity to the protease of SEQ ID NO: 1, which is derived from Nocardiopsis sp. NRRL 18262, and described in WO 88/03947 and WO 01/58276.

Additional proteases of the invention are disclosed in WO 2004/111220, WO 2004/111221, WO 2004/111222, WO 2004/111223, WO 2005/035747, WO 2004/111219, hereby incorporated by reference.

Particular examples of proteases of the invention are derived from Nocardiopsis dassonvillei subsp. dassonvillei DSM 43235 (SEQ ID NO: 2), Nocardiopsis alba DSM 15647 (SEQ ID NO: 3), Nocardiopsis prasina DSM 15648 (SEQ ID NO: 4), Nocardiopsis prasina DSM 15649 (SEQ ID NO: 5), as well as fragments, mutants, and variants thereof, such as Protease 22 (SEQ ID NO: 6). Optionally, each of SEQ ID NOs: 1-6 has a C-terminal extension consisting of one or more amino acids, for example non-polar or uncharged amino acids, such as one or more of Q, S, V, A, or P, preferably selected from the group consisting of: QSHVQSAP (SEQ ID NO:7), QSAP, QP, TL, TT, QL, TP, LP, TI, IQ, QP, PI, LT, TQ, IT, QQ, and PQ.

In particular embodiments, the proteases of the invention are selected from the group consisting of:
(a) proteases belonging to the EC 3.4 . . . enzyme group;
(b) serine proteases;
(c) serine proteases of peptidase family S2A;
(d) serine proteases of peptidase family S1E as described in Biochem. J. 290:205-218 (1993) and in MEROPS protease database, release 6.20, Mar. 24, 2003, (www.merops.ac.uk). The database is described in Rawlings, N.D., O'Brien, E. A. & Barrett, A. J. (2002) MEROPS: the protease database. Nucleic Acids Res. 30, 343-346; and
(e) proteases derived from strains of Nocardiopsis.

For determining whether a given protease is a serine protease, and a family S2A protease, reference is made to the above Handbook and the principles indicated therein. Such determination can be carried out for all types of proteases, be it naturally occurring or wild-type proteases; or genetically engineered or synthetic proteases.

In particular embodiments, the degree of identity to SEQ ID NO: 1 is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments, the degree of identity is at least about 50%, 51%, 52%, 53%, 54%, 55%, 56%, 57%, 58%, 59%, 60%, 61%, 62%, 63%, 64%, 65%, 66%, 67%, 68%, or at least 69%.

In further particular embodiments, the protease of the invention is acid-stable, which means that the protease activity of the pure protease enzyme, in a dilution corresponding to A₂₈₀=1.0, and following incubation for 2 hours at 37° C. in the following buffer: 100 mM succinic acid, 100 mM HEPES, 100 mM CHES, 100 mM CABS, 1 mM CaCl₂, 150 mM KCl, 0.01% Triton® X-100, pH 3.5; is at least 40% (or at least 45, 50, 55, 60, 65, 70, 75, 80, 85, 90, 95, or at least 97%) of the reference activity, as measured using the assay described in Example 2C of WO 01/58276 (substrate: Suc-AAPF-pNA, pH 9.0, 25° C.). The term reference activity refers to the protease activity of the same protease, following incubation in pure form, in a dilution corresponding to A₂₈₀=1.0, for 2 hours at 5° C. in the following buffer: 100 mM succinic acid, 100 mM HEPES, 100 mM CHES, 100 mM CABS, 1 mM CaCl₂, 150 mM KCl, 0.01% Triton® X-100, pH 9.0, wherein the activity is determined as described above. The term A₂₈₀=1.0 means such concentration (dilution) of said pure protease which gives rise to an absorption of 1.0 at 280 nm in a 1 cm path length cuvette relative to a buffer blank. The term pure protease refers to a sample with a A₂₈₀/A₂₆₀ ratio above or equal to 1.70 (see Example 2E of WO 01/58276), and which by a scan of a Coomassie stained SDS-PAGE gel is measured to have at least 95% of its scan intensity in the band corresponding to said protease (see Example 2A of WO 01/58276).

In still further particular embodiments, optionally, an additional protease may be used, for example a mammalian protease, for example in the form of pancreas extract from swine, or a microbial protease, for example derived from bacterial or fungal strains, such as Bacillus, Pseudomonas, Aspergillus, or Rhizopus. The protease may in particular be derived from a strain of Aspergillus, such as Aspergillus oryzae or Aspergillus melleus, in particular the product Prozyme 6™ (neutral, alkaline protease EC 3.4.21.63) which is commercially available from Amano Pharmaceuticals, Japan.

The protease of the invention may be used in combination with a lipase.

In the present context, a lipase means a carboxylic ester hydrolase EC 3.1.1.-, which includes activities such as EC 3.1.1.3 triacylglycerol lipase, EC 3.1.1.4 phospholipase A1, EC 3.1.1.5 lysophospholipase, EC 3.1.1.26 galactolipase, EC 3.1.1.32 phospholipase A1, EC 3.1.1.73 feruloyl esterase. In a particular embodiment, the lipase is an EC 3.1.1.3 triacylglycerol lipase.

In particular embodiments, the lipase is a mammalian lipase, for example in the form of pancreas extract from swine, or a microbial lipase, for example derived from bacterial or fungal strains, such as Bacillus, Pseudomonas, Aspergillus, or Rhizopus. The lipase may in particular be derived from a strain of Rhizopus, such as Rhizopus javanicus, Rhizopus oryzae, or Rhizopus delemar, for example the product Lipase D Amano 2000™ (also designated Lipase D2™) which is commercially available from Amano Pharmaceuticals, Japan.

In further particular embodiments, the lipase for use in the present invention is a recombinantly produced microbial lipase, for example derived from a fungus such as Humicola or Rhizomucor, from a yeast such as Candida, or from a bacterium such as Pseudomonas. In a preferred embodiment, the lipase is derived from a strain of Humicola lanuginosa or Rhizomucor miehei.

The Humicola lanuginosa (synonym Thermomyces lanuginosus) lipase (SEQ ID NO: 8) is described in EP 305216, and particular lipase variants are described in, for example, WO 92/05249, WO 92/19726, WO 94/25577, WO 95/22615, WO 97/04079, WO 97/07202, WO 99/42566, WO 00/32758, WO 00/60063, WO 01/83770, WO 02/055679, and WO 02/066622. Still further examples of fungal lipases are the cutinase from Humicola insolens which is described in EP 785994, and the phospholipase from Fusarium oxysporum which is described in EP 869167. Examples of yeast lipases are lipase A and B from Candida antarctica of which lipase A is described in EP 652945, and lipase B is described by, for example, Uppenberg et al in Structure, 2 (1994), 293. An example of a bacterial lipase is the lipase derived from Pseudomonas cepacia, which is described in EP 214761.

In a preferred embodiment, the lipase is at least 70% identical to the lipase of SEQ ID NO: 8. In additional preferred embodiments, the degree of identity to SEQ ID NO: 8 is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments, the degree of identity to SEQ ID NO: 8 is at least about 50%, 51%, 52%, 53%, 54%, 55%, 56%, 57%, 58%, 59%, 60%, 61%, 62%, 63%, 64%, 65%, 66%, 67%, 68%, or at least 69%.

In a still further preferred embodiment, the lipase, like the mammalian pancreatic lipase, is a 1,3-position specific lipase.

The protease of the invention, with or without a lipase as described above, may also be used in combination with an amylase.

In the present context, an amylase is an enzyme that catalyzes the endo-hydrolysis of starch and other linear and branched oligo- and polysaccharides. The amylose part of starch is rich in 1,4-alpha-glucosidic linkages, while the amylopectin part is more branched containing not only 1,4-alpha- but also 1,6-alpha-glucosidic linkages. In a particular embodiment, the amylase is an enzyme belonging to the EC 3.2.1.1 group.

In particular embodiments, the amylase is a mammalian amylase, for example in the form of pancreas extract from swine, or a microbial amylase, for example derived from bacterial or fungal strains, such as Bacillus, Pseudomonas, Aspergillus, or Rhizopus.

The amylase may in particular be derived from a strain of Aspergillus, such as Aspergillus niger, Aspergillus oryzae or Aspergillus melleus, for example either of the products Amylase A1™ derived from Aspergillus oryzae which is commercially available from Amano Pharmaceuticals, Japan, or Amylase EC™ derived from Aspergillus melleus which is commercially available from Extract-Chemie, Germany.

Other examples of fungal amylases are the Aspergillus niger amylase (SWISSPROT P56271), which is also described in Example 3 of WO 89/01969, and the Aspergillus oryzae amylase (SEQ ID NO: 9). Examples of variants of the Aspergillus oryzae amylase are described in WO 01/34784.

The alpha-amylase derived from Bacillus licheniformis is an example of a bacterial alpha-amylase. This amylase is, for example, described in WO 99/19467, together with other homologous bacterial alpha-amylases derived from, for example, Bacillus amyloliquefaciens, and Bacillus stearothermophilus, as well as variants thereof. Examples of additional amylase variants are those described in U.S. Pat. No. 4,933,279; EP 722490, and EP 904360.

In a particular embodiment, the amylase is at least 70% identical to the amylase of SEQ ID NO: 9. In additional preferred embodiments, the degree of identity to SEQ ID NO: 9 is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments, the degree of identity to SEQ ID NO: 9 is at least about 50%, 51%, 52%, 53%, 54%, 55%, 56%, 57%, 58%, 59%, 60%, 61%, 62%, 63%, 64%, 65%, 66%, 67%, 68%, or at least 69%.

Generally, the protease, lipase, and amylase enzymes (hereinafter “the enzyme(s)”) for use according to the invention may be natural or wild-type enzymes obtained from animals, in particular mammals, for example human or swine enzymes; from plants, or from microorganisms, but also any mutants, variants, fragments etc. thereof exhibiting the desired enzyme activity, as well as synthetic enzymes, such as shuffled enzymes, and consensus enzymes.

In a specific embodiment, the enzyme(s) are low-allergenic variants, designed to invoke a reduced immunological response when exposed to animals, including man. The term immunological response is to be understood as any reaction by the immune system of an animal exposed to the enzyme(s). One type of immunological response is an allergic response leading to increased levels of IgE in the exposed animal. Low-allergenic variants may be prepared using techniques known in the art. For example the enzyme(s) may be conjugated with polymer moieties shielding portions or epitopes of the enzyme(s) involved in an immunological response. Conjugation with polymers may involve in vitro chemical coupling of polymer to the enzyme(s), e.g. as described in WO 96/17929, WO 98/30682, WO 98/35026, and/or WO 99/00489. Conjugation may in addition or alternatively thereto involve in vivo coupling of polymers to the enzyme(s). Such conjugation may be achieved by genetic engineering of the nucleotide sequence encoding the enzyme(s), inserting consensus sequences encoding additional glycosylation sites in the enzyme(s) and expressing the enzyme(s(in a host capable of glycosylating the enzyme(s), see e.g. WO 00/26354. Another way of providing low-allergenic variants is genetic engineering of the nucleotide sequence encoding the enzyme(s) so as to cause the enzymes to self-oligomerize, effecting that enzyme monomers may shield the epitopes of other enzyme monomers and thereby lowering the antigenicity of the oligomers. Such products and their preparation is described e.g. in WO 96/16177. Epitopes involved in an immunological response may be identified by various methods such as the phage display method described in WO 00/26230 and WO 01/83559, or the random approach described in EP 561907. Once an epitope has been identified, its amino acid sequence may be altered to produce altered immunological properties of the enzyme(s) by known gene manipulation techniques such as site directed mutagenesis (see e.g. WO 00/26230, WO 00/26354 and/or WO 00/22103) and/or conjugation of a polymer may be done in sufficient proximity to the epitope for the polymer to shield the epitope.

In particular embodiments, the protease, lipase, and/or amylase enzymes are (i) stable at pH 4-8, preferably also at pH 3-4, more preferably at pH 3.5; (ii) active at pH 4-9, preferably 4-8, more preferably at pH 6.5; (iii) stable against degradation by pepsin and other digestive proteases (such as pancreas proteases, i.e., mainly trypsin and chymotrypsin); and/or (iv) stable and/or active in the presence of bile salts

The term “in combination with” refers to the combined use according to the invention of the protease, lipase, and/or amylase. The combined use can be simultaneous, overlapping, or sequential, these three terms being generally interpreted in the light of the prescription made by the physician.

The term “simultaneous” refers to circumstances under which the enzymes are active at the same time, for example when they are administered at the same time as one or more separate pharmaceutical products, or if they are administered in one and the same pharmaceutical composition.

The term “sequential” refers to such instances where one and/or two of the enzymes are acting first, and the second and/or third enzyme subsequently. A sequential action can be obtained by administering the enzymes in question as separate pharmaceutical formulations with desired intervals, or as one pharmaceutical composition in which the enzymes in question are differently formulated (compartmentalized), for example with a view to obtaining a different release time, providing an improved product stability, or to optimizing the enzyme dosage.

The term “overlapping” refers to such instances where the enzyme activity periods are neither completely simultaneous nor completely sequential, viz. there is a certain period in which the enzymes are both, or all, active.

The term “a”, for example when used in the context of the protease, lipase, and/or amylase of the invention, means at least one. In particular embodiments, “a” means “one or more,” or “at least one”, which again means one, two, three, four, five etc.

For purposes of the present invention the degree of identity between two amino acid sequences is determined by the program “align” which is a Needleman-Wunsch alignment (i.e. a global alignment). The sequences are aligned by the program, using the default scoring matrix BLOSUM50 is used. The penalty for the first residue of a gap is 12, and for further residues of a gap the penalties are 2.

“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

The activity of the enzyme(s) of the invention can be measured using any suitable assay. Generally, assay-pH and assay-temperature are to be adapted to the enzyme in question. Examples of assay-pH-values are pH 2, 3, 4, 5, 6, 7, 8, 9, 10, 11, or 12. Examples of assay-temperatures are 30, 35, 37, 40, 45, 50, 55, 60, 65, 70, 80, 90, or 95° C.

For example, protease activity can be measured using any assay, in which a substrate is employed, that includes peptide bonds relevant for the specificity of the protease in question.

Examples of suitable enzyme assays are included in the experimental part. Other examples are the Ph.Eur. assays for lipase and amylase activity.

### Medicament

In the present context, the term “medicament” means a compound, or mixture of compounds, that treats, prevents and/or alleviates the symptoms of disease. The medicament may be prescribed by a physician, or it may be an over-the-counter product.

### Pharmaceutical Compositions

Isolation, purification, and concentration of the enzyme(s) of the invention may be carried out by conventional means.

In a particular embodiment, concentrated solid or liquid preparations of each of the enzyme(s) are prepared separately. These concentrates may also, at least in part, be separately formulated, as explained in more detail below.

In a further particular embodiment, the enzyme(s) are incorporated in the pharmaceutical compositions of the invention in the form of solid concentrates. The enzyme(s) can be brought into the solid state by various methods as is known in the art. For example, the solid state can be either crystalline, where the enzyme molecules are arranged in a highly ordered form, or a precipitate, where the enzyme molecules are arranged in a less ordered, or disordered, form.

Crystallization may, for example, be carried out at a pH close to the pl of the enzyme(s) and at low conductivity, for example 10 mS/cm or less, as described in EP 691982 (see also Example 2 herein).

Various precipitation methods are known in the art, including precipitation with salts, such as ammonium sulphate, and/or sodium sulphate; with organic solvents, such as ethanol, and/or isopropanol; or with polymers, such as PEG (Poly Ethylene Glycol). In the alternative, the enzyme(s) can be precipitated from a solution by removing the solvent (typically water) by various methods known in the art, e.g. lyophilization, evaporation (for example at reduced pressure), and/or spray drying.

In a further particular embodiment, the solid concentrate of the enzyme(s) has a content of active enzyme protein of at least 50% (w/w) by reference to the total protein content of the solid concentrate. In still further particular embodiments, the content of active enzyme protein, relative to the total protein content of the solid concentrate is at least 55, 60, 65, 70, 75, 80, 85, 90, or at least 95% (w/w). The protein content can be measured as is known in the art, for example using a commercial kit, such as Protein Assay ESL, order no. 1767003, which is commercially available from Roche, or on the basis of the method described in Example 8 of WO 01/58276.

A pharmaceutical composition of the invention comprises the enzyme(s), preferably in the form of concentrated enzyme preparations, more preferably solid concentrates, together with at least one pharmaceutically acceptable auxiliary, or subsidiary, material such as (i) at least one carrier and/or excipient; or (ii) at least one carrier, excipient, diluent, and/or adjuvant. Non-limiting examples of, optional, other ingredients, all pharmaceutically acceptable, are disintegrators, lubricants, buffering agents, moisturizing agents, preservatives, flavouring agents, solvents, solubilizing agents, suspending agents, emulsifiers, stabilizers, propellants, and vehicles.

Generally, depending i.a. on the medical indication in question, the composition of the invention may be designed for all manners of administration known in the art, including enteral administration (through the alimentary canal), and parenteral administration, for example by injection (such as subcutaneous, intramuscular, or intravenous, etc.). Thus, the composition may be in solid, semi-solid, liquid, or gaseous form, such as tablets, capsules, powders, granules, microspheres, ointments, creams, foams, solutions, suppositories, injections, inhalants, gels, microspheres, lotions, and aerosols.

The following methods and auxiliary materials are merely exemplary and are in no way limiting.

For solid oral preparations, the enzyme(s) can be used alone or in combination with appropriate additives to make tablets, powders, granules or capsules, for example, with conventional carriers, such as lactose, mannitol, corn starch, or potato starch; with excipients or binders, such as crystalline, or microcrystalline, cellulose, cellulose derivatives, acacia, corn starch, or gelatins; with disintegrators, such as corn starch, potato starch, or sodium carboxymethylcellulose; with lubricants, such as carnauba wax, white wax, shellac, waterless colloid silica, macrogol 6000, povidone, talc, monoolein, or magnesium stearate; and if desired, with diluents, adjuvants, buffering agents, moistening agents, preservatives such as methylparahydroxybenzoate (E218), colouring agents such as titanium dioxide (E171), and flavouring agents such as saccharose, saccharin, orange oil, lemon oil, and vanillin. Oral preparations are examples of preferred preparations for treatment of the medical indication of PEI.

The enzyme(s) can also, quite generally, be formulated into preparations for injection, or into liquid oral preparations, by dissolving, suspending, or emulsifying them in an aqueous solvent such as water, or in non-aqueous solvents such as vegetable or other similar oils, synthetic aliphatic acid glycerides, esters of higher aliphatic acids, propylene glycol, polyethylene glycol such as PEG 4000, or lower alcohols such as linear or ramified C1-C4 alcohols, for example 2-propanol; and if desired, with conventional subsidiary materials or additives such as solubilizers, adjuvants, diluents, isotonic agents, suspending agents, emulsifying agents, stabilizers, and preservatives.

The enzyme(s) can furthermore, still quite generally, be utilized in aerosol formulation to be administered via inhalation, for example by formulation into pressurized acceptable propellants such as dichlorodifluoromethane, propane, nitrogen, and the like.

Furthermore, the enzyme(s) can generally be made into suppositories for rectal administration by mixing with a variety of bases such as emulsifying bases or water-soluble bases. The suppository can include vehicles such as cocoa butter, carbowaxes and polyethylene glycols, which melt at body temperature, yet are solidified at room temperature.

The use of liposomes as a delivery vehicle is another method of possible general interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like. For preparing the liposomes, the procedure described by Kato et al. (1991) J. Biol. Chem. 266:3361 may be used.

Unit dosage forms for oral or rectal administration such as syrups, elixirs, powders, and suspensions may be provided wherein each dosage unit, for example, teaspoonful, tablespoonful, capsule, tablet or suppository, contains a predetermined amount of the enzyme(s). Similarly, unit dosage forms for injection or intravenous administration may comprise the enzyme(s) in a composition as a solution in sterile water, normal saline, or another pharmaceutically acceptable carrier.

The term “unit dosage form”, as used herein, refers to physically discrete units suitable as unitary dosages for human and animal subjects, each unit containing a predetermined quantity of enzyme(s) in an amount sufficient to produce the desired effect.

In a particular embodiment, the pharmaceutical composition of the invention is for enteral, preferably oral, administration.

In further particular embodiments, the oral composition is (i) a liquid composition containing crystals of the enzyme(s); (ii) a liquid suspension of sediments of (highly) purified enzyme(s); (iii) a gel containing the enzyme(s) in solid or solubilized form; (iv) a liquid suspension of immobilized enzyme(s) or of enzymes adsorbed to particles and the like; or (v) a solid composition in the form of enzyme(s)-containing powder, pellets, granules, or microspheres, if desired in the form of tablets, capsules, or the like, that are optionally coated, for example with an acid-stable coating.

In another particular embodiment of the composition, the enzyme(s) are compartmentalized, viz. separated from each other, for example by means of separate coatings.

In a still further particular embodiment of the composition, the protease is separated from other enzyme components of the composition, such as the lipase, and/or the amylase.

The dosage of the enzyme(s) will vary widely, depending on the specific enzyme(s) to be administered, the frequency of administration, the manner of administration, the severity of the symptoms, and the susceptibility of the subject to side effects, and the like. Some of the specific enzymes may be more potent than others.

The amide (peptide) bonds, as well as the amino and carboxy termini, may be modified for greater stability on oral administration. For example, the carboxy terminus may be amidated.

### Methods of Treatment

The protease of the invention, optionally in combination with a lipase, and/or an amylase (the enzyme(s) of the invention), is useful in the therapeutic, and/or prophylactic, treatment of various diseases or disorders in animals. The term “animal” includes all animals, and in particular human beings. Examples of animals are non-ruminants, and ruminants, such as sheep, goats, horses, and cattle, e.g. beef cattle, cows, and young calves. In a particular embodiment, the animal is a non-ruminant animal. Non-ruminant animals include mono-gastric animals, e.g. pigs or swine (including, but not limited to, piglets, growing pigs, and sows); poultry such as turkeys, ducks and chicken (including but not limited to broiler chicks, layers); young calves; pets such as cat, and dog; and fish (including but not limited to salmon, trout, tilapia, catfish and carps; and crustaceans (including but not limited to shrimps and prawns). In a particular embodiment the animal is a mammal, more in particular a human being.

For example, the enzyme(s) are useful in the treatment of digestive disorders like maldigestion or dyspepsia that are often caused by a deficient production and/or secretion into the gastrointestinal tract of digestive enzymes normally secreted from, i.a., the stomach, and the pancreas.

Further, the enzyme(s) are particularly useful in the treatment of PEI. PEI can be verified using, i.a., the Borgström test (JOP. J Pancreas (Online) 2002; 3(5):116-125), and it may be caused by diseases and conditions such as pancreatic cancer, pancreatic and/or gastric surgery, e.g. total or partial resection of the pancreas, gastrectomy, post gastrointestinal bypass surgery (e.g. Billroth II gastroenterostomy); chronic pancreatitis; Shwachman Diamond Syndrome; ductal obstruction of the pancreas or common bile duct (e.g. from neoplasm); and/or cystic fibrosis (an inherited disease in which a thick mucus blocks the ducts of the pancreas). The enzyme(s) may also be useful in the treatment of acute pancreatitis.

The effect of the enzyme(s) on digestive disorders can be measured as generally described in EP 0600868, in which Example 2 describes an in vitro digestibility test for measuring lipase stability test under gastric conditions, and Example 3 an in vitro digestibility test for lipase activity in the presence of bile salts. Corresponding tests can be set up for the protease and amylase. Also WO 02/060474 discloses suitable tests, for example (1) an in vitro test for measuring lipid digestion in a swine test feed, and (2) an in vivo trial with pancreas insufficient swine in which the digestibility of fat, protein and starch is measured.

In a particular embodiment, the effect of the protease of the invention is measured using the in vitro pancreas insufficiency digestion model of Example 1 herein, in which various other substrates may be used as desired, for example animal protein, other vegetable proteins, cereals, animal or vegetable fats and oils, as well as any mixtures thereof.

In other particular embodiments, the effect of the protease of the invention is measured using the in vivo screening test for protease efficacy of Example 4, or the full in vivo digestibility trial of Example 5.

As another example, the enzyme(s) are useful in the treatment of Diabetes mellitus type I, and/or type II, in particular for adjuvant treatment in a diabetes therapy of digestive disorders usually accompanying this disease, with a view to diminishing late complications.

The effect on Diabetes mellitus of the enzyme(s) may be determined by one or more of the methods described in WO 00/54799, for example by controlling the level of glycosylated haemoglobin, the blood glucose level, hypoglycemic attacks, the status of fat-soluble vitamins like vitamins A, D and E, the required daily dosage of insulin, the body-weight index, and hyper glycaemic periods.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

### Example 1

### In Vitro Pancreatic Insufficiency Digestion Model

A purified preparation of the protease derived from Nocardiopsis sp. NRRL 18262 (SEQ ID NO: 1) was prepared as generally described in Example 2 of WO 01/58276, and tested in an in vitro model simulating the digestion in individuals suffering from pancreatic insufficiency.

The in vitro system consists of 24 flasks in which a substrate (based on maize and soybean meal (SBM)) was initially incubated with HCl/pepsin (simulating gastric digestion), and subsequently with two reduced levels of pancreatin, simulating intestinal digestion in an individual with partial and complete pancreatic insufficiency. A positive control experiment was also included with a normal level of pancreatin.

10 of the flasks were dosed with the protease at the start of the gastric phase whereas the remaining flasks served as blanks. At the end of the intestinal incubation phase samples of in vitro digesta were removed and analysed for solubilised and digested protein.

### Outline of In Vitro Digestion Procedure

| | | | Time | Simulated digestion |
|---|---|---|---|---|
| Components added | pH | Temperature | course | phase |
| 10 g maize/-SBM substrate | 3.0 | 40° C. | t = 0 min | Mixing |
| (6:4), 41 ml HCl (0.105M) | | | | |
| 5 ml HCl (0.105M)/pepsin | 3.0 | 40° C. | t = 30 min | Gastric digestion |
| (3000 U/g substrate), 100 mg | | | | |
| protease EP/kg substrate) | | | | |
| 16 ml H₂O | 3.0 | 40° C. | t = 1.0 hour | Gastric digestion |
| 7 ml NaOH (0.39M) | 6.8 | 40° C. | t = 1.5 hours | Intestinal digestion |
| 5 ml NaHCO₃ (1M)/ | 6.8 | 40° C. | t = 2.0 hours | Pancreatin-defective |
| pancreatin (0, 4 or 8 mg/g | | | | intestinal digestion |
| substrate) | | | | (plus a positive |
| | | | | control) |
| Terminate incubation | 7.0 | 40° C. | t = 6.0 hours | |

### Conditions

Substrate: 4 g SBM, 6 g maize (premixed)
HCl: 0.105 M for 1.5 hours (i.e. 30 min HCl-substrate premixing)
pepsin: Sigma P-7000; 3000 U/g substrate for 1 hour
pancreatin: Sigma P-7545; 0, 4, or 8 mg/g substrate for 4 hours (the assumed normal level of pancreatin being 8 mg/g)
protease: 100 mg protease enzyme protein (EP)/kg of substrate (Enzyme Protein was calculated on the basis of the A₂₈₀ values and the amino acid sequences (amino acid compositions) using the principles outlined in S. C. Gill & P. H. von Hippel, Analytical Biochemistry 182, 319-326, (1989))
pH: 3.0 stomach step/6.8-7.0 intestinal step
temperature: 40° C.
Replicates: 5 (4)

### Solutions

### 0.39 M NaOH

### 0.105 M HCl

0.105 M HCl containing 6000 U pepsin per 5 ml
1 M NaHCO₃ containing 16 mg pancreatin per ml

### 125 mM NaAc-buffer, pH 6.0

### Experimental Procedure for In Vitro Model

The experimental procedure was according to the above outline. pH was measured at time 1, 2.5, and 5.5 hours. Incubations were terminated after 6 hours and samples of 30 ml were removed and placed on ice before centrifugation (10000×g, 10 min, 4° C.). Supernatants were removed and stored at −20° C.

### Analysis

All samples were analysed for content of solubilised and digested protein using gel filtration.

### Estimation of Solubilised and Digested Protein

The content of solubilised protein in supernatants from in vitro digested samples was estimated by quantifying crude protein (CP) using gel filtration HPLC. Supernatants were thawed, filtered through 0.45 μm polycarbonate filters and diluted (1:50, v/v) with H₂O. Diluted samples were chromatographed by HPLC using a Superdex Peptide PE (7.5×300 mm) gel filtration column (Global). The eluent used for isocratic elution was 50 mM sodium phosphate buffer (pH 7.0) containing 150 mM NaCl. The total volume of eluent per run was 26 ml and the flow rate was 0.4 ml/min. Elution profiles were recorded at 214 nm and the total area under the profiles was determined by integration. To estimate protein content from integrated areas, a calibration curve (R²=0.9993) was made from a dilution series of an in vitro digested reference maize/-SBM sample with known total protein content. The protein determination in this reference sample was carried out using a standard method (in this case the Kjeldahl method for determination of % nitrogen; A.O.A.C. (1984) Official Methods of Analysis 14th ed., Washington D.C.).

The content of digested protein was estimated by integrating the chromatogram area corresponding to peptides and amino acids having a molecular mass of 1500 Dalton or below (Savoie, L.; Gauthier, S. F. Dialysis Cell For The In-vitro Measurement Of Protein Digestibility. J. Food Sci. 1986, 51, 494-498; Babinszky, L.; Van, D. M. J. M.; Boer, H.; Den, H. L. A. An In-vitro Method for Prediction of The Digestible Crude Protein Content in Pig Feeds. J. Sci. Food Agr. 1990, 50, 173-178; Boisen, S.; Eggum, B. O. Critical Evaluation of In-vitro Methods for Estimating Digestibility in Simple-Stomach Animals. Nutrition Research Reviews 1991, 4, 141-162). To determine the 1500 Dalton dividing line, the gel filtration column was calibrated using cytochrome C (Boehringer, Germany), aprotinin, gastrin 1, and substance P (Sigma Aldrich, USA), as molecular mass standards.

The experimental results are shown in Table 1 and also visualized in FIG. 1. In Table 1, the column “Enzyme” shows the amount of pancreatin (abbreviated “pan”) per gram of substrate, as well as the amount of the Nocardiopsis protease (in square brackets). In the next column, “n” is the number of replicates of each experiment. The following cluster of columns shows the percentage of digestible Crude Protein (abbreviated % dig.CP), including the Standard Deviation (SD), and the significance superscript letter as explained in the footnote below the table. And finally, the last cluster of columns shows the percentage of soluble Crude Protein (abbreviated % sol.CP), also including SD and significance superscript letters.

As it appears from Table 1, there is a strong tendency (P<0.10) for the addition of the Nocardiopsis protease to improve the percentage of solubilized, as well as digestible protein, and this is so in the experiment with 4 mg/g pancreatin, as well as in the experiment with 0 mg/g pancreatin (compare “4 mg pan, [100]” with “4 mg pan, [0];” and “0 mg pan, [100]” with “0 mg pan, [0]”). This means that the Nocardiopsis protease is able to compensate for the partial or complete absence of pancreatin.

TABLE 1

| | | Of total protein | | | |
|---|---|---|---|---|---|
| Enzyme [mg EP/kg] | N | % dig. CP | SD | % sol. CP | SD |
| 8 mg pan, [0] | 5 | 54.7 | ^{C}3.0 | 98.2 | ^{C}4.0 |
| 4 mg pan, [0] | 5 | 47.0 | ^{A}2.2 | 88.1 | ^{B}4.0 |
| 0 mg pan, [0] | 4 | 45.1 | ^{A}4.1 | 83.8 | ^{A}2.4 |
| 4 mg pan, [100] | 5 | 55.0 | ^{C}1.2 | 95.8 | ^{C}1.8 |
| 0 mg pan, [100] | 5 | 49.9 | ^{B}0.7 | 88.8 | ^{B}1.2 |

LSD 90%
Values within a column with different capital superscript letters indicate a strong tendency for a difference (1-way ANOVA, Least Significant Difference (LSD) test, P < 0.10). SD = Standard Deviation.

### Example 2

### Preparation of Crystallized Protease Preparations

The protease of SEQ ID NO: 1 was fermented as described in Example 1, and the protease-containing broth was harvested on a centrifuge at pH 4.5. The resulting supernatant was subjected to ultra-filtration using a membrane with a cut-off value of 6 kDal, and to diafiltration until a conductivity of 2 mS/cm in the protease-containing solution. The content of protease was approximately 100 mg/mL.

The concentrated and diafiltered protease solution is crystallized by adjusting pH with sodium hydroxide to pH 8.5, i.e. close to the pl of the protease (which is 9.3). After pH adjustment the solution is left over night at room temperature, and crystallization takes place.

The following day the crystallized protease is harvested by centrifugation.

### Example 3

### Enzyme Assays

### Protease

### Substrate: Suc-MPF-pNA (Sigmas S-7388).

Assay buffer: 100 mM succinic acid, 100 mM HEPES, 100 mM CHES, 100 mM CABS, 1 mM CaCl₂, 150 mM KCI, 0.01% Triton®X-100 adjusted to pH 9.0 with HCl or NaOH.
Assay emperature: 25° C.

300 μl diluted protease sample was mixed with 1.5 ml of the assay buffer and the activity reaction was started by adding 1.5 ml pNA substrate (50 mg dissolved in 1.0 ml DMSO and further diluted 45× with 0.01% Tritone X-100) and, after mixing, the increase in A405 was monitored by a spectrophotometer as a measurement of the protease activity. The protease samples were diluted prior to the activity measurement in order to ensure that all activity measurements fell within the linear part of the dose-response curve for the assay.

### Protease FIP Assay

Protease activity may also be determined using the FIP assay (Fédération Internationale Pharmaceutique), 1 FIP-unit=1 Ph.Eur.-unit (European Pharmacopoeia). This assay is described, together with other FIP assays in: Fédération Internationale Pharmaceutique, Scientific Section: International Commission for the standardisation of pharmaceutical enzymes. a) “Pharmaceutical Enzymes,” Editors: R. Ruyssen and A. Lauwers, E. Story Scientia, Ghent, Belgium (1978), b) European Pharmacopoeia. See also Deemester et al in Lauwers A, Scharpé S (eds): Pharmaceutical Enzymes, New York, Marcel Dekker, 1997, p. 343-385.

Principle: The substrate casein is hydrolysed by protease at pH 7.5 and at a temperature of 35° C. The reaction is stopped by addition of trichloroacetic acid, and non-degraded casein is filtered off. The quantity of peptides remaining in solution is determined by spectrophotometry at 275 nm.

Definition of the activity: The protease activity is determined as the quantity of peptides not precipitated by a 5.0% (wt/vol, i.e. 5.0 g/100 ml) solution of trichloroacetic acid, by reference to a pancreas reference powder (protease reference standard) of known FIP activity.

### Materials and Methods:

Casein Solution:

1.25 g casein (dry matter), e.g. Calbiochem no. 218680, is suspended in water until a practically clear solution is obtained. pH is adjusted to 8.0, and the solution is diluted with water to a final volume of 100 ml. Here and in the following, water means deionized water.

Borate Buffer pH 7.5:

2.5 g sodium chloride, 2.85 g disodium tetraborate and 10.5 g boric acid are dissolved in 900 ml water, pH is adjusted to pH 7.5+/−0.1 and diluted to 1000 ml with water.

Filter Paper:

Folded filters with a diameter of 125 mm, e.g. Schleicher & Schuell no. 1573½. Test of filter paper: Filter 5 ml of 5.0% trichloro acetic acid through the filter. The absorption at 275 nm of the filtrate should be less than 0.04, using unfiltered trichloroacetic acid solution as a blank.

Protease Reference Standard:

Protease (pancreas) commercially available from the International Commission on Pharmaceutical Enzymes, Centre for Standards, Harelbekestraat 72, B-9000 Ghent, Belgium. The standard has a labelled activity (A) in FIP/Ph.Eur.-units/g. Accurately weigh a quantity corresponding to approx. 130 protease-FIP/Ph.Eur.-units. Add a spatula tip of sea sand, wet with a few drops of ice-cold 0.02M calcium chloride (pH 6.0-6.2), and triturate the whole with a flat-ended glass rod. Dilute with approx. 90 ml of the same ice-cold calcium chloride solution and stir the suspension for 15 to 30 minutes in an ice-bath. pH is adjusted to 6.1 and the volume is adjusted to 100 ml with the same calcium chloride solution. 5.0 ml of this suspension is diluted with borate buffer pH 7.5 to 100 ml. For the activity test, 1.0, 2.0 and 3.0 ml of this solution is used as reference (in what follows designated S1, S2, and S3, S for Standard).

Test Suspension:

Prepare a suspension of the sample as described above for the protease reference standard, using a sample amount equivalent to approx. 260 FIP/Ph.Eur.-units. pH is adjusted to 6.1 and water is added to 100 ml. 5.0 ml of this solution is mixed with 5 ml of calcium chloride solution. 5 ml of this dilution is further diluted to 100 ml with borate buffer. Use 2.0 ml of this solution for the assay (in what follows the sample is designated Un, sample of unknown activity, number n).

Assay Procedure (Activity Test):

The assay is performed for the three reference suspensions (S1, S2, S3) and for the sample suspension (Un), all in triplicate. One blank per sample is sufficient (designated S1b, S2b, S3b, and Unb, respectively). A blind (B) is prepared without without sample/standard as compensation liquid for the spectrophotometer. Borate buffer is added to tubes as follows: Blind (B) 3.0 ml; sample (Un) 1.0 ml; standards (S1, S2 and S3) 2.0, 1.0 and 0 ml, respectively. Protease reference standard is added to S1, S2 and S3 as follows: 1.0, 2.0, and 3.0 ml, respectively. The test suspension is added to the sample tubes as follows (Un): 2.0 ml. 5 ml trichloro acetic acid is added to all blinds (S1b, S2b, S3b, Unb and B) followed by immediate mixing. All tubes are stopped with a glass stopper and placed together with the substrate solution in a water-bath at constant temperature (35+/−0.5° C.). When temperature equilibration is reached, at time zero, 2.0 ml casein solution is added to tubes S1, S2, S3 and Un, followed by immediate mixing. Exactly 30 minutes after, 5.0 ml. trichloro acetic acid is added to each of tubes S1, S2, S3 and Un, followed by immediate mixing. The tubes are withdrawn from the water bath and allowed to stand at room temperature for 20 minutes to complete the precipitation of the proteins. The content of each tube is filtered twice through the same filter, and the absorption of the filtrates is measured at 275 nm using the filtrate from tube B as compensation liquid. The activity of the sample (Un) in FIP units is calculated relative to the known labelled activity (A) of the standards (S1, S2, S3). The absorption values minus the respective blinds (e.g. the absorption of S1 minus the absorption of S1b) should lie in the interval of 0.15-0.60.

### Lipase

Substrate: para-Nitro-Phenyl (pNP) Valerate

### Assay pH: 7.7

Assay temperature: 40° C.
Reaction time: 25 min

The digested product with yellow colour has a characteristic absorbance at 405 nm. Its quantity is determined by spectrophotometry. One lipase unit is the amount of enzyme which releases 1 micromole titratable butyric acid per minute under the given assay conditions. A more detailed assay description, AF95/6-GB, is available on request from Novozymes A/S, Krogshoejvej 36, DK-2880 Bagsvaerd, Denmark.

### Amylase

Substrate: Phadebas tablets (Pharmacia Diagnostics; cross-linked, insoluble, blue-coloured starch polymer, which is mixed with bovine serum albumin and a buffer substance, and manufactured into tablets)

### Assay Temperature: 37° C.

### Assay pH: 4.3

Reaction time: 20 min

After suspension in water the starch is hydrolyzed by the alpha-amylase, giving soluble blue fragments. The absorbance of the resulting blue solution, measured at 620 nm, is a function of the alpha-amylase activity. One Fungal alpha-Amylase Unit (1 FAU) is the amount of enzyme which breaks down 5.26 g starch (Merck, Amylum soluble Erg. B. 6, Batch 9947275) per hour at the standard assay conditions. A more detailed assay description, APTSMYQI-3207, is available on request from Novozymes A/S, Krogshoejvej 36, DK-2880 Bagsvaerd, Denmark.

### Example 4

### In Vivo Screening Test for Protease Efficacy

The protease described in Example 1 was tested in female Göttingen minipigs (Ellegaard). In the minipigs, the pancreatic duct was ligated to induce Pancreatic Exocrine Insufficiency (PEI), and they were fitted with an ileo-caecal re-entrant cannula, all under halothane anaesthesia and at a weight of about 25 kg, as described in Tabeling et al., J. 1999, Studies on nutrient digestibilities (pre-caecal and total) in pancreatic duct-ligated pigs and the effects of enzyme substitution, J. Anim. Physiol. A. Anim. Nutr. 82: 251-263 (hereinafter referred to as “Tabeling 1999”); and in Gregory et al., J. 1999. Growth and digestion in pancreatic duct ligated pigs, Effect of enzyme supplementation in “Biology of the Pancreas in Growing Animals” (S G Pierzynowski & R. Zabielski eds), Elsevier Science BV, Amsterdam, pp 381-393 (hereinafter referred to as “Gregory et al 1999”). A period of at least 4 weeks was allowed for recovery from surgery, before studies were commenced. Prior to study begin, the PEI status of each pig was confirmed via the stool chymotrypsin test (commercially available from Immundiagnostik AG, Wiesenstrasse 4, D-64625 Bensheim, Germany, with catalogue No. K 6990).

During the studies, the pigs were housed in modified metabolism cages on a 12:12 h light-dark cycle and allowed free access to water and fed two meals/day. To assess protease efficacy, the pigs were fed a 250 g test meal mixed with 1 liter of water, 0.625 g Cr₂O₃ (chromic oxide marker) and into which differing amounts of protease (0, 1000, 2500, 6000 FIP U protease/meal (protease FIP units, see Example 3)) were mixed immediately before feeding. The test meal contained 21.4% protein, 51.9% starch, 2.6% fat, and had the following composition (g/100 g dry matter): Fish meal 3.5, poultry meat meal 10.2, wheat flour 29.5, shelled rice 14, potato starch 11, maize starch 14, casein 5.9, cellulose powder 4.3, vitamins, minerals and trace elements 7.6 (as per the nutritional requirement for pigs/piglets, see e.g. Table A of WO 01/58276).

Ileal chyme was collected on ice for a total of 8 h after first appearance of the meal marker in the ileum (green chyme) and stored at −20° C. before analysis. At least one day washout was allowed between separate determinations.

In brief, the frozen samples were freeze-dried and analysed for dry matter (DM) and crude protein. DM was estimated by weight after freeze-drying followed by 8 h incubation at 103° C. Crude protein was calculated as nitrogen (N) multiplied by a factor 6.25, i.e. Crude protein (g/kg)=N (g/kg)×6.25 as stated in Animal Nutrition, 4th edition, Chapter 13 (Eds. P. McDonald, R. A. Edwards and J. F. D. Greenhalgh, Longman Scientific and Technical, 1988, ISBN 0-582-40903-9). The nitrogen content was determined by the Kjeldahl method (Naumann and Bassler, 1993, Die chemische Untersuchung von Futtermitteln. 3 edition VDLUFA-Verlag, Darmstadt, Germany (VDLUFA=Verband Deutscher Landwirtschaftlicher Untersuchungs-und Forschungsanstalten).

Calculation of apparent pre-caecal protein digestibility was made according to the formula:

$$Apparent digestibility ( \% ) = 100 - [ \frac{\% {Cr}_{2} {O}_{3} in feed}{\% {Cr}_{2} {O}_{3} in sample} \cdot \frac{\% protein in sample}{\% protein in feed} \cdot 100 ]$$

in which Cr₂O₃ and protein were expressed as g/100 g dry matter.

TABLE 2
Influence of enzyme supplementation
on apparent protein digestibility

| Enzyme | | | | |
|---|---|---|---|---|
| Supplement | 0 | 1000 FIP U | 2500 FIP U | 6000 FIP U |
| No supplement | 14.7 ± 2.1 | | | |
| Pancreatin | | 31.7 ± 12.4 | 59.4 ± 4.9 | 70.7 ± 0.9 |
| Protease | | 55.2 ± 4.3 | 61.7 ± 4.8 | 68.4 ± 3.9 |

Values are mean ± SD

### Example 5

### Full In Vivo Digestibility Trial

The protease described in Example 1 was tested in female Göttingen minipigs (Ellegaard) in which the pancreatic duct was ligated to induce PEI, and they were fitted with an ileo-caecal re-entrant cannula, all under halothane anaesthesia and at a weight of about 25 kg, as previously described (Tabeling 1999; Gregory et al 1999). Control minipigs were prepared in similar manner, but the pancreatic duct was left intact. A period of at least 4 weeks was allowed, for recovery from surgery, before studies were commenced. Prior to study begin, the PEI status of each pig was confirmed via the stool chymotrypsin test (see Example 4).

The pigs were allowed free access to water and fed two 250 g meals/day, at 08.00 and 20.00 h, of a finely milled diet (as in Example 4), mixed with 1 litre water, 0.625 g Cr₂O₃ and into which differing amounts of protease (0, 6000 FIP U protease/meal) were mixed immediately before feeding. Each dose was fed to the pigs for 2 weeks and ileal chyme was collected on ice for 12 h for the final 3 days. The samples were stored at −20° C. until analysis.

In brief, the frozen samples were freeze-dried and analysed for dry matter (DM) and crude protein. DM and crude protein was estimated and pre-caecal protein digestibility (apparent digestibility) calculated as described in Example 4.

Pre-caecal protein digestibility was ca. 80% in control (pancreatic sufficient) minipigs on the diet used. In the untreated PEI minipig, protein digestibility was severely reduced compared to these control values, but enzyme supplementation with pancreatin or the microbial protease strongly improved digestibility, which approached control values (see the results in Table 3 below).

TABLE 3
Influence of enzyme supplementation
on apparent protein digestibility:

| | 0 Enzymes | 6000 U/meal |
|---|---|---|
| Control | 81.3 ± 2.6 | — |
| PEI untreated | 30.5 ± 6.8 | — |
| PEI + pancreatin | — | 71.5 ± 4.1 |
| PEI + Protease | — | 65.3 ± 0.7 |

Values are mean ± SD

1. A protease of at least 70% identity to SEQ ID NO: 1, for use as a medicament.

2-6. (canceled)

7. A pharmaceutical composition comprising a protease of at least 70% identity to SEQ ID NO: 1, preferably in the form of a solid concentrate, together with at least one pharmaceutically acceptable auxiliary material.

8. The composition of claim 7, further comprising a lipase or an amylase, preferably in the form of a solid concentrate.

9. The composition of claim 7, further comprising a lipase and an amylase, preferably in the form of solid concentrates.

10. A method for the treatment of digestive disorders, pancreatic insufficiency, pancreatitis, cystic fibrosis, diabetes type I, and/or diabetes type II, by administering a therapeutically effective amount of a protease of at least 70% identity to SEQ ID NO: 1.

11. The method of claim 10, further comprising administering a therapeutically effective amount of a lipase or an amylase.

12. The method of claim 10, further comprising administering a therapeutically effective amount of a lipase and an amylase.
